(19) United States
(12) Patent Application Publication
(10) Pub. No.: US 2021/0393971 A1
(43) Pub. Date: Dec. 23, 2021

(54) TRANSCRANIAL MAGNETIC STIMULATION TREATMENT MONITORING AND NOTIFICATION

(71) Applicant: United States Government as Represented by the Department of Veterans Affairs, Washington, DC (US)

(72) Inventor: Punit Vaidya, Cleveland, OH (US)

(21) Appl. No.: 17/350,748

(22) Filed: Jun. 17, 2021

Related U.S. Application Data
(60) Provisional application No. 63/040,323, filed on Jun. 17, 2020.

(51) Int. Cl. A61N 2/02

(52) U.S. Cl. CPC A61N 2/02

(57) ABSTRACT

Provided herein are methods and systems for determining and presenting information related to transcranial magnetic stimulation (TMS) treatment. A system comprises a transcranial magnetic stimulation coil and an array of electrical contacts, and a first device configured to determine information related to the TMS treatment and transmit the information to at least one additional device configured to receive and display the information.

### CROSS REFERENCE TO RELATED PATENT APPLICATION

This application claims priority to U.S. Provisional Application No. 63/040,323 filed Jun. 17, 2020, the entirety of which is herein incorporated by reference.

### BACKGROUND

Repetitive transcranial magnetic stimulation (TMS) is a noninvasive form of brain stimulation that can be used for treatment of major depressive disorder or other conditions in those who have not responded to medications. The treatment involves using an insulated conducting coil that generates strong magnetic pulses to regulate activity in areas of the brain underlying the coil. Patients are typically seated in a chair, awake and alert during the treatments. Standard treatment regimens require a patient to sit still for the duration of the 20 to 40 minutes treatment session, often four or five days a week for several weeks or months. Standard TMS equipment provides little to no information to a patient regarding the status of a treatment session. Further, in TMS devices that include a user interface, the user interface is inadequate for viewing from more than a few feet away and sometimes does not provide important information about the treatment. The disclosure recognizes and addresses, amongst other technical challenges, the lack of adequate user interfaces to monitor treatment delivered by existing TMS devices, and the issue of poor patient experience during such treatment.

### SUMMARY

It is to be understood that both the following general description and the following detailed description is merely an example and is explanatory only and is not restrictive. Methods, systems, and apparatuses for transcranial magnetic stimulation treatment monitoring and notification are described. TMS involves several important variables related to TMS pulses (e.g., electromagnetic pulses) such as pulse frequency, pulses per train, inter-train interval, total pulses delivered, combinations thereof, and the like. While the term TMS is used throughout, it is understood that TMS refers to all forms of TMS including rTMS, dTMS, etc . . . TMS pulses produce loud clicking sounds and strong, sometimes uncomfortable, tapping sensations on the scalp of a patient. In some embodiments of the disclosure, audio signals associated with the clicking sounds, and digital signals associated with the electromagnetic pulses, individually or in combination, can be used to provide a patient and/or a TMS operator with nearly real-time information about the status of a TMS treatment session. Embodiments of the technologies of this disclosure provide a remote monitoring device configured to determine various parameters of a current TMS treatment session and can retain a record of one or more previous treatment sessions. Embodiments of the disclosure include a TMS treatment monitoring device configured to optionally output audible and/or visible information regarding the progress of the TMS treatment session. Embodiments of the disclosure include a portable and wireless TMS treatment monitoring device configured to be utilized by a TMS operator. Embodiments of the disclosure provide a method for TMS pulse data and, based on the TMS pulse data, sending one or more signals.

A treatment notification device may be configured to provide unobtrusive information to a patient about the progress of the TMS treatment session (e.g., time remaining in the treatment session, information about pulse cycles etc.). The treatment notification device may also be configured to provide, in some cases, audible and/or visual alerts of forthcoming (e.g., inbound) TMS pulses, one or more pulse cycles, or an end of (e.g., a termination of) a TMS session, combinations thereof, and the like. The treatment monitoring device can provide a useful method for the TMS operator/clinician to track current and past TMS sessions delivered in clinical or research settings. The treatment monitoring device can be used with any TMS equipment. The technologies disclosed herein can improve the patient experience and can monitor any TMS equipment used for clinical or research purposes, without limitation to one particular manufacturer and/or equipment model. The treatment monitoring technologies of this disclosure may be configured to operate in an unobtrusive manner (e.g., in the background) of a TMS treatment. The systems and apparatuses may be configured to operate automatically (e.g., without a user input).

Additional features or advantages of the disclosed technologies will be set forth in part in the description which follows, and in part will be apparent from the description, or may be learned by practice of this disclosure. The advantages of the disclosure can be realized and attained by means of the elements and combinations particularly pointed out in the appended claims. It is to be understood that both the foregoing general description and the following detailed description are exemplary and explanatory only and are not restrictive of the subject disclosure.

This summary is not intended to identify critical or essential features of the disclosure, but merely to summarize certain features and variations thereof. Other details and features will be described in the sections that follow.

### BRIEF DESCRIPTION OF THE DRAWINGS

The accompanying drawings, which are incorporated in and constitute a part of this specification, together with the description, serve to explain the principles of the methods and systems:

FIG. 1A shows an example TMS treatment room

FIG. 1B shows an example TMS treatment environment;

FIG. 1C shows an example TMS treatment environment;

FIG. 2A shows an example TMS treatment monitoring device;

FIG. 2B shows an example TMS treatment monitoring device;

FIG. 3A shows an example of a graphical interface;

FIG. 3B shows an example of a graphical interface;

FIG. 3C shows an example of a graphical interface;

FIG. 4A shows an example of a TMS treatment notification device;

FIG. 4B shows an example of a TMS treatment notification device;

FIG. 5 shows an example method; and

FIG. 6 shows an example system.

### DETAILED DESCRIPTION

Before the present TMS systems and techniques are disclosed and described, it is to be understood that this disclosure is not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude other components, integers or steps. “Exemplary” means “an example of” and is not intended to convey an indication of a preferred or ideal configuration. “Such as” is not used in a restrictive sense, but for explanatory purposes.

Disclosed are components that can be used to perform the disclosed TMS systems and techniques. These and other components are disclosed herein. It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

This detailed description may refer to a given entity performing some action. It should be understood that this language may in some cases mean that a system (e.g., a computer) owned and/or controlled by the given entity is actually performing the action.

This detailed description may use certain terms such as “up,” “down,” “upper,” “lower,” “upward,” “downward,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise.

Additionally, instances in this detailed description where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

A plurality of TMS pulses may be administered. The one or more TMS pulses may be administered rapidly and/or in sets which can be spaced out at intervals of approximately 11 to approximately 26 seconds. The one or more TMS pulses may produce or otherwise be associated with loud clicking sounds. A computing device may be configured to receive or otherwise detect or determine the one or more pulses. For example, the computing device may comprise an audio module configured to detect sound (e.g., analog sound waves in the air including ambient noise, sounds associated with the plurality of TMS pulses, combinations thereof, and the like). The computing device may be configured to detect, based on the sound, the plurality of TMS pulses. For example, the computing device may be configured to execute an audio processing algorithm to detect the presence of the one or more TMS pulses within an environment (e.g., a TMS treatment room) as described in more detail below. The computing device may be configured to determine TMS pulse data associated one or more TMS pulses of the plurality of TMS pulses. The computing device may send, for example to a display device or some other device, one or more signals. The one or more signals may comprise the TMS data and other TMS information and may be output audibly and/or visually for observation by a patient or TMS operator. For example, a wall-mounted unit may be configured to receive the one or more signals and output visual and/or audible alerts to the patient regarding the status of the TMS treatment, such as time left in treatment, or notification of an upcoming set of TMS pulses to reduce startling the patient For example, the treatment monitoring technologies can determine date/time of treatment, total number of pulses administered, treatment duration, interval between sets of pulses, etc. The monitoring device may be configured to facilitate manual control for starting, pausing, and stopping a treatment cycle. The monitoring device may be configured to facilitate changing a default total number of pulses and an intertrain interval (e.g., a time between pulses and/or trains of pulses).

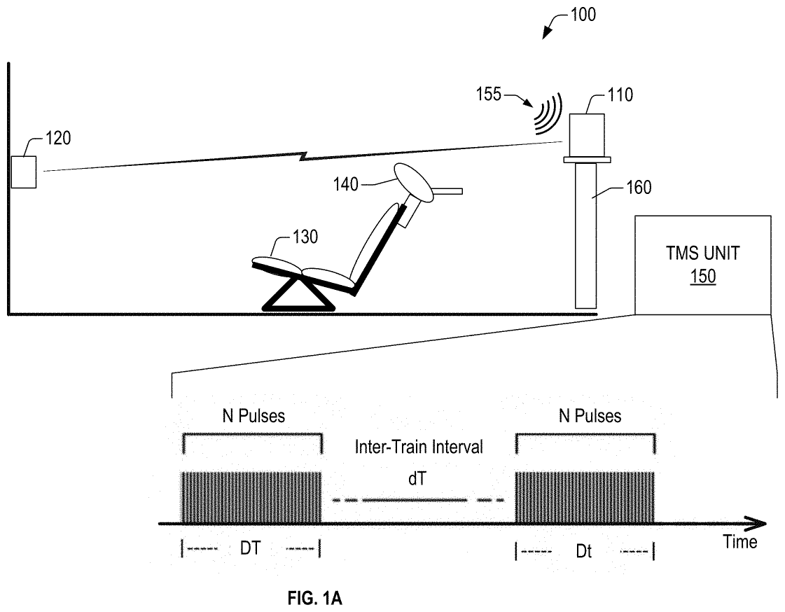

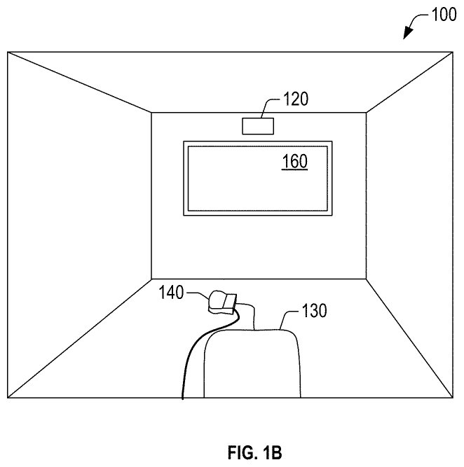

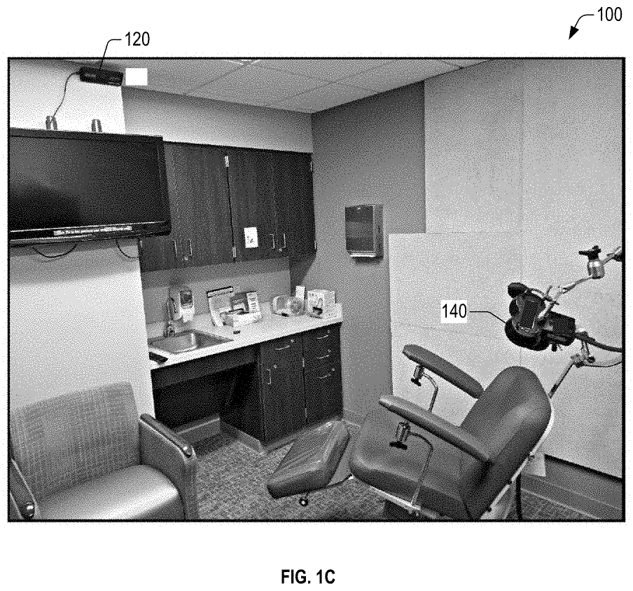

With reference to the drawings, FIG. 1A shows an example of a TMS treatment room 100 (or other treatment environment). The treatment room 100 may comprise a treatment monitoring device 110 and a treatment notification device 120, in accordance with one or more embodiments of this disclosure. The TMS treatment room 100 may comprise a TMS treatment chair 130 where a patient (not depicted in FIG. 1A) can receive a TMS treatment. The TMS treatment room 100 may comprise one or more coils 140 placed near a TMS unit 150. For example, a TMS device may comprise may comprise the one or more coils 140. The TMS unit 150 may comprise circuitry configured to provide magnetic stimulation. For example, the TMS unit 150 may comprise a TMS pulse generator (not shown in FIG. 1A). The one or more coils 140 may be configured for electromagnetic induction. For example, the one or more coils may be configured to generate a rapidly changing magnetic field, of the order of tens of thousands of Tesla/second. The nature of the rapidly changing magnetic field may produce a focused electrical field in the brain of the patient. The focused electrical field in the brain mayo depolarize neurons underlying the one or more coils 140. During a treatment session, such a magnetic field is applied as a train of N TMS pulses (N being a natural number) that span a particular interval ΔT (e.g., four seconds). The magnitude of N may be determined by a frequency f of generation of the TMS pulses and ΔT (e.g., f=10 Hz, ΔT=4 s, and N=40). After the train of TMS pulses ends, there is a pause in the application of the magnetic field and then a next train of TMS pulses is applied. The next train of TMS pulses also can span the particular interval. The pause between a current train of pulses and the next train of pulses spans a defined period referred to as the inter-pulse interval. The inter-pulse interval δT can have a magnitude in a range from about 11 s to about 26 s.

Each TMS pulse in the train of TMS pulses (e.g., the plurality of TMS pulses) can be brief and can have a large amplitude. For example, a TMS pulse of the plurality of TMS pulses can last (e.g., span temporally) approximately 100 μs to 200 μs, and can have an amplitude of approximately 1.5 Tesla. As a result, the TMS pulse can create a transient deformation of the one or more coils 140, which can in turn produce a loud cracking sound (e.g., a pulse sound). Therefore, a TMS treatment session usually includes a plurality of pulse sounds associated with the plurality of TMS pulses.

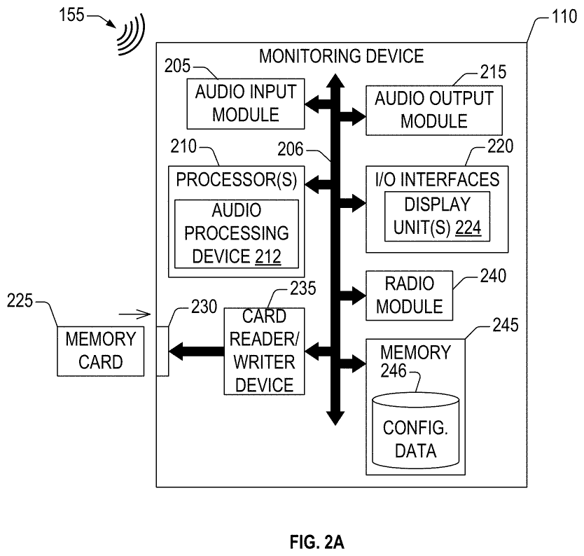

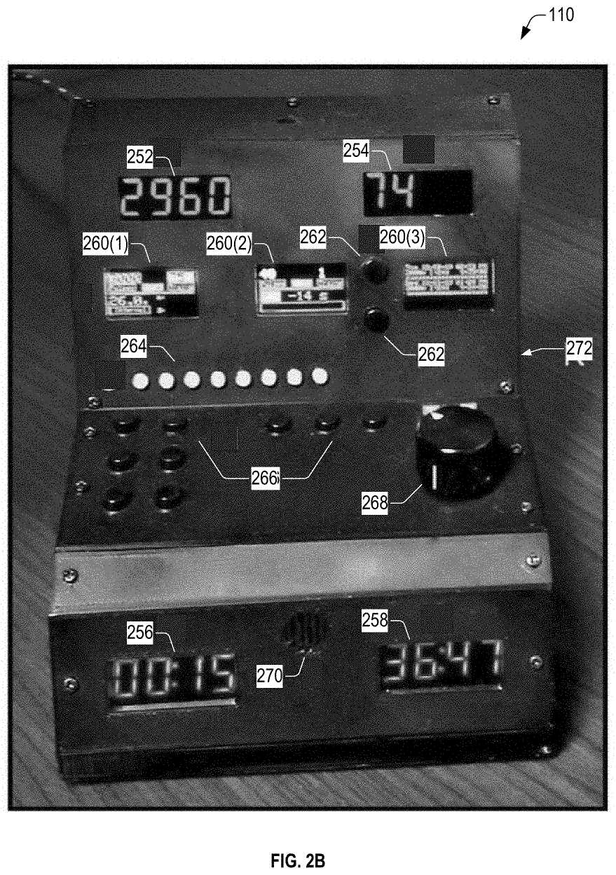

A pulse sound can measure up to 140 dB near the one or more coils 140 and may comprise one or more dominant frequency components in a range from approximately 2 kHz to about 5 kHz. The treatment monitoring device 110 may be configured to determine a characteristic sound profile of one or more TMS pulses of the plurality of TMS pulses. Based on the characteristic sound profile of the one or more TMS pulses, the treatment monitoring device 110 may distinguish the sound of the TMS pulse from other sounds, such as ambient noise, speech, utterances, music, etc. Accordingly, the treatment monitoring device 110 can detect the application of a TMS pulse by monitoring ambient sound 155 within the treatment room 100. As is described in greater detail below, the treatment monitoring device 110 may be configured to apply essentially continuous audio processing to the ambient sound 155 to determine if and when a TMS pulse has been emitted. By aurally detecting the one or more TMS pulses, the treatment monitoring device 110 can determine and record several parameters of associated with individual TMS pulses and/or an entire treatment session comprising a series of TMS pulse trains. As an example, the treatment monitoring device 110 can determine treatment date/start time, number of pulses delivered, inter-train interval, duration of treatment, time until next set of pulses, time left in treatment, treatment end time, a combination thereof, or similar. To determine time, the treatment monitoring device 110 can include a clock, such as a VCO, real time clock (RTC) and counter(s) circuitry (neither depicted in FIG. 2A).

The treatment monitoring device 110 may be configured to send and/or receive data to and/or from the treatment notification device 110. The data may comprise or otherwise be associated with one or more of the foregoing parameters of a TMS treatment session. The treatment monitoring device 110 also can send signaling to the treatment notification device 110. The treatment monitoring device 110 and the treatment notification device 120 may be configured for wireless and/or wired communication. The signaling can include, for example, configuration instructions, control instructions, and/or or other types of commands. The commands may comprise signal, for example the current time, time left in treatment, time until next pulse, time remaining for any given aspect of treatment, combinations thereof, and the like. For example, the signals may comprise, or cause, audio signals and/or visual signals. To send data and signaling wirelessly from to the treatment notification device 120, the treatment monitoring device 110 can include a radio module.

The treatment monitoring device 110 may comprise a radio module. The radio module, in accordance with aspects of this disclosure, can operate in a variety of wireless environments having wireless signals conveyed in different electromagnetic radiation (EM) frequency bands. To that end, the radio module can include one or more antennas and a communication processing unit that can process (code, decode, format, etc.) wireless signals within a set of one or more EM frequency bands (also referred to as frequency bands (e.g., 2.4 GHz band) comprising one or more of radio frequency (RF) portions of the EM spectrum, microwave portion(s) of the EM spectrum, or infrared (IR) portion of the EM spectrum. In one aspect, the set of one or more frequency bands can include at least one of (i) all or most licensed EM frequency bands, or (ii) all or most unlicensed frequency bands currently available for telecommunication. A combination of receiving (RX) antenna(s) and at least a portion of the communication processing unit can constitute a receiver of the radio module. The communication processing circuitry can include coder(s), decoder(s), multiplexer(s), demultiplexer(s), and similar components. A combination of transmitting (TX) antenna(s) and at least a portion of the communication processing unit can constitute a transmitter of the radio module. Transmitter and receiver form a transceiver of the radio module. Such a radio module can operate according to a communication mode determined by a radio protocol. In some cases, the radio module can permit wireless communication according to a point-to-point radio protocol, such as Bluetooth, Zigbee, or similar.

The treatment monitoring device 110 can be portable and can have one of various form factors. For example, as is illustrated in FIG. 1A, the treatment monitoring device 110 can rest on a desktop, a pedestal 160, or another type of rigid based. In some embodiments, the treatment monitoring device 110 can be a desktop apparatus. In other embodiments, the treatment monitoring device can be a handheld device (e.g., a tablet computer).

The treatment notification device 120 also can have one of various form factors. As is illustrated in FIG. 1A, the treatment notification device 120 can be wall-mounted within the treatment room 100, in a position within line-of-sight (LOS) from the patient. In some cases, as is shown in FIG. 1B, the treatment notification device 120 can be placed above a television set 160, within a LOS from the patient. The television set 160 also can be wall-mounted within the TMS treatment room 100. The treatment notification device 110 also can be placed in other positions near the television set 160. FIG. 1C illustrates another example of a TMS treatment room 100, including the treatment notification device 120, the television set 160, the TMS chair 130, and the coil(s) 140. In other cases, the treatment notification device 120 can be a handheld apparatus (e.g., a tablet computer) that can be provided to the patient receiving treatment.

The treatment notification device 120 can receive data wirelessly from the treatment monitoring device 110. The treatment notification device 120 also can receive signaling wirelessly from the treatment monitoring device 110. The signaling can include, for example, configuration instructions, control instructions, and/or or other types of commands. To receive data and signaling wirelessly from the treatment monitoring device 110, the treatment notification device 120 also includes a radio module. The radio module can operate in a similar fashion as the radio module integrated into the treatment monitoring device 110.

FIG. 2A is a schematic block diagram of the treatment monitoring device 110, in accordance with one or more embodiments of this disclosure. As described herein, the treatment monitoring device 110, by means of the audio processing device 212, can distinguish sound caused by a TMS pulse from other sounds, such as background noises from a television, speech, or other noises (e.g., keystrokes, mouse clicks, door knocks, taps of a pen on a desk, and similar). To that end, the treatment monitoring device 110 can include an audio input module 205 that can receive the ambient sound 155. The ambient sound may comprise the one or more TMS pulse noises as well as other sounds. The audio input module 215 can include microphone(s), analog-to-digital converter(s), amplifier(s), filter(s), and/or other circuitry for processing of audio (e.g., equalizer(s)). As such, in some cases, a microphone included in the audio input module 215 can receive the ambient sound 155. The audio input module 205 also can generate an ambient audio signal representative of the ambient sound 155. The ambient audio signal can be an analog signal (e.g., such as a soundwave in air).

The audio input module 205 can provide the audio signal to one or more processor(s) 210 included in the monitoring device 110. The processor(s) 210 can be arranged in numerous configurations depending at least on the specific complexity (functionality, operational capacity, etc.) of the notification device 120. Accordingly, the processor(s) 120 can be embodied in, or can constitute, a central processing unit (CPU); multiple CPUs; a graphics processing unit (GPU); multiple GPUs; a microprocessor or another type of digital signal processor; a programmable logic controller (PLC); a programmable microcontroller; an ASIC; a FPGA; other types of processing circuitry for executing program code or performing defined operations; a combination thereof; or similar.

To send the audio signal to at least one of the processor(s) 210, the audio input module 205 can be connected to the processor(s) 210 by means of a bus architecture 206, for example. The processor(s) can include a digital-to-analog converter (DAC; not depicted in FIG. 2A) and/or an analog-to-digital converter, that can generate a digital audio signal using the ambient audio signal. The DAC may be used by the notification system to, for example, play alert sounds or otherwise output one or more notifications or information. The processor(s) 210 also can include an audio processing device 212 that can essentially continuously analyze the digital audio signal for sound corresponding to TMS pulses. The audio processing device 212 can implement an audio processing algorithm that can accurately detect, within the digital audio signal, the characteristic sound produced by the TMS pulse.

The audio processing device 212 can analyze the audio signal representative of the sound 155 nearly continuously. The audio processing device 212 can detect the one or more TMS pulses nearly as they occur. Detection of the one or more TMS pulses can result in a record of a time at which the one or more TMS pulses are detected. Thus, the audio processing device 212 can generate a series of time offsets that permit characterizing a TMS session. As an example, if the TMS session is started, the treatment monitoring device 110 (via the audio processing device 212, for example) can detect an initial train of 40 pulses of a TMS pulse train at 10 Hz (e.g., an initializing pulse train). The treatment monitoring device 110 can thus determine that a TMS session has started. The treatment monitoring device 110 can include one or more memory devices 245 (referred to as memory 245) retaining configuration data 246 defining one or many TMS treatments. The memory 245 can include one or more non-transitory storage media. In some embodiments, the memory 245 includes one or several solid-state memory devices.

At least some of those treatments can correspond to FDA-approved treatments for respective neurological ailments, for example. Other TMS treatments retained in the memory 245 can correspond to other types of therapies besides FDA-approved treatments. Using the detected first treatment pulse train and the configuration data 246, the monitoring treatment device 110 can determine that 3,000 pulses are likely to be administered, as per a standard protocol for the treatment of depression. As another example, if the initially detected TMS pulses occur at 18 Hz for about 2 seconds, as used in some TMS equipment, the treatment monitoring device 110 can determine a different set of treatment parameters, such as 1980 total pulses per treatment session. The configuration data 246 also can retained such a set of treatment parameters.

It is noted that the monitoring device 110 need not pre-determine a total number of expected pulses pertaining to a TMS treatment session. In some configurations, the treatment monitoring device 110 can continue detecting pulses until a termination criterion is satisfied. For example, the at least one of the processor(s) 210 can direct the audio processing device 212 to terminate processing an audio signal after a period of one or more inter-pulse intervals has elapsed without detection of a TMS pulse. One or more termination criteria can be retained in the memory 245, for example. In other configurations, the treatment monitoring device 110 can receive input data defining the number of TMS pulses to be administered during a current TMS session.

The treatment monitoring device 110 may detect a second treatment pulse train. Upon detecting a second treatment pulse train of TMS pulses, the treatment monitoring device 110 can determine an inter-train interval. The treatment monitoring device 110 can use the inter-train interval to interpolate the duration of the entire treatment session or time remaining until completion of the treatment session, or both. In one configuration, at least one of the processor(s) 210 can determine the inter-train interval, the duration of treatment session, and time remaining until completion of the treatment session.

The treatment monitoring device 110 also can include an audio output module 215. In some embodiments, the audio output module 215 can include a group of audio output devices (e.g., a headphone socket or piezoelectric speaker) and circuitry that permit generating and sending audio output signal (noise, tones, utterances, speech, music, and the like). Such circuitry can include, in some cases, digital-to-analog converters; volume control(s) and/or other audio controls.

Based on the inter-train interval determination, at least one of the processor(s) 210 can direct the audio output module 215 to emit an audible sound of a defined duration to indicate the application of a forthcoming set of TMS pulses. In one example, the audible sound can be customized sound. To that end, at least one of the processor(s) 210 can execute a custom sound file. In some embodiments, the custom sound file can be retained in the memory 245. In other embodiments, the custom sound filed can be retained in a memory card 225 (e.g., a microSD card) coupled to a card interface and connector 235. In those embodiments, the treatment monitoring device 110 can include a card reader device 235 that can load the customs sound file to memory 245 or system memory (not depicted in FIG. 2A) available to the processor(s) 210. The treatment monitoring device 110 also can detect the completion of the treatment and, in response, also can indicate such a completion with a second audible sound of a defined duration. It is noted that in conventional TMS equipment, audible signals indicating the completion of a treatment session are not provided to an operator or patient.

The disclosure is not limited to audible sounds to indicate a forthcoming set of TMS pulses and/or the completion of a TMS session. In some embodiments, the monitoring treatment device 110 can include multiple input/output (I/O) interfaces 220. One or many of the I/O interfaces 220 can provide visible signals indicating a forthcoming set of TMS pulses and/or the completion of the TMS session. In some embodiments, the I/O interfaces 220 can include a group of color LED devices that can display an unobtrusive signal to convey a forthcoming set of pulses or the completion of the TMS session. The unobtrusive signal can be, for example, a brief pulsing light, a particular combination of colors, a particular combination of illuminated LED(s) and non-illuminated LED(s), or similar.

The treatment monitoring device 110 can demarcate the end of a treatment session in response to one or many conditions being satisfied. In one example, the end of the treatment session can be demarcated after the completion of an rTMS session, which can be auto-detected based on having detected 3,000 pulses or another number of pulses consistent with another magnetic stimulation protocol). In another example, the end of the treatment session can be demarcated in an instance in which there is a sufficient time since a last detected TMS pulse (e.g., 5 minutes, which would be the typical minimum time needed between patients. Regardless of the particular termination condition that is satisfied, demarcating the end of the treatment session can include storing parameters of the treatment session in a memory device. In some embodiments, the treatment monitoring device 110 can store such parameters in a memory card 225 present in the treatment monitoring device 110. For instance, the card reader/writer device 235 can append the parameters to a text file stored onto the memory card 225 (e.g., a microSD card). Examples of such parameters include day, date, and time of day at the start of the treatment, total pulses administered, inter-train interval, and duration of the treatment.

Additional or alternative operations can be implemented in response to termination of the treatment session. In some embodiments, the I/O interfaces 220 can include one or many display unit(s) 224. At least one of the display unit(s) 224 can present parameters of a treatment session that has been completed. Information about past treatments can be readily reviewed by a TMS operator at any date by accessing those parameters via the display unit(s) 224 and/or other I/O interfaces integrated into the treatment monitoring device 110.

Additionally, or in some embodiments, the monitoring device 110 can include multiple sensors (not depicted in FIG. 2A) to detect ambient room conditions, such as temperature, humidity, and light levels, or other conditions that may impact the coils.

TMS stimulation intensity is another useful parameter that may be detected. To that end, a calibration with a particular TMS device and treatment setting may be implemented, as sound amplitude caused by a TMS pulse can increase as the stimulus output of the TMS coil 140 (FIG. 1) is increased, but differs between coils. The audio processing device 212 can utilized such a calibration to determine presence of a TMS pulse within the ambient sound 155 (FIG. 1).

FIG. 2B shows an example treatment monitoring device 110 in accordance with one or more embodiments of this disclosure. As is illustrated, the display unit(s) 224 (FIG. 2A) can include a first display unit 252 having multiple 7-segment LED display devices that can present the number of TMS pulses remaining in the session. The treatment notification unit 120 also includes a second display unit 254 also having multiple 7-segment LED display devices that can present the number of TMS pulse trains remaining in the session.

In addition, the treatment monitoring device 110 can include a third display unit 256 having multiple 7-segment LED display devices that can present the time elapsed since the start of the current TMS session in minutes and seconds, for example. The treatment monitoring device 110 can include a forth display unit 258 having multiple 7-segment LED display devices that can present the time left in the current session in minutes and seconds, for example.

As is illustrated in FIG. 2B, the display unit(s) 224 (FIG. 2A) also can include a first display device 260(1), a second display device 260(2), and third display device 260(3). In an embodiment, the first display 260(1), 260(2), and 260(3) may comprise a single display. Each one of those devices is oriented towards the TMS operator in order to convey the information related to current and past TMS treatment sessions.

Examples of those display devices include an LCD or LED display device, or similar. An LED display device can be an organic LED (OLED) display device. The disclosure is, of course, not limited to an array of several display devices. Indeed, in some cases, a single, larger touchscreen display device can be integrated into the treatment monitoring device 110 instead of such an array.

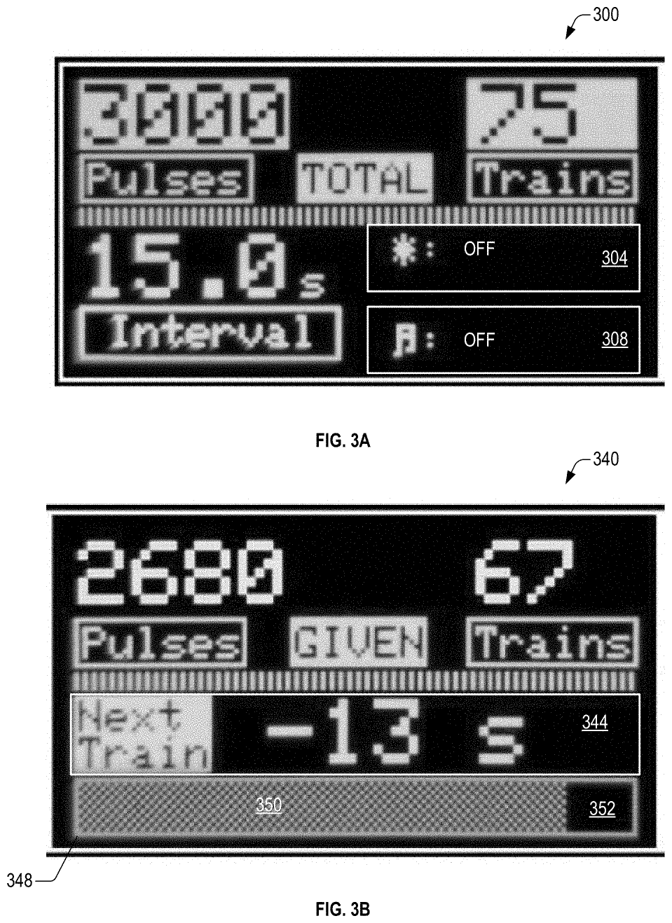

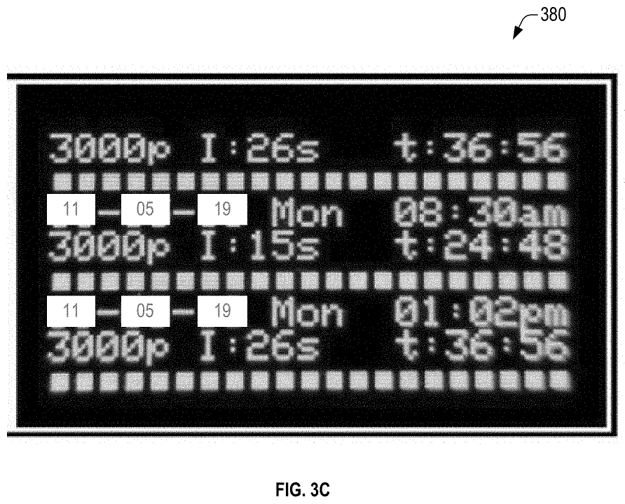

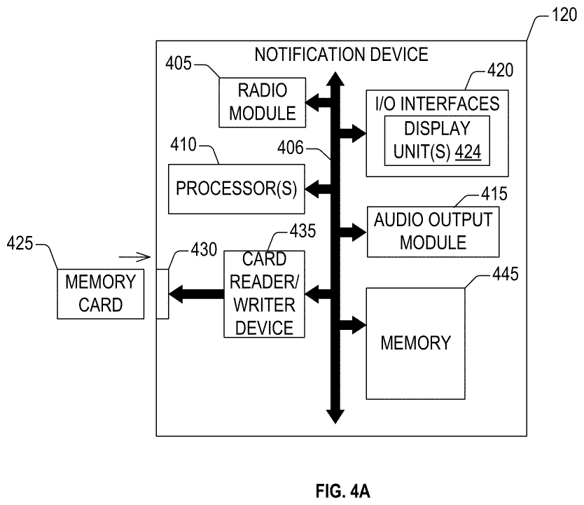

FIG. 3A shows an example of a graphical interface 300 that one of the display devices 260(1) to 260(3) can display in connection with a TMS treatment. The graphical interface 300 presents the total number of TMS pulses and pulse trains for a treatment session. In this example, the treatment monitoring device 110 may have detected the first set of pulses at 10 Hz lasting 4 seconds, and thus, the treatment monitoring device 110 can determine that the standard FDA-approved protocol for depression is being administered. Such protocol consists of 3000 pulses per session divided in 75 trains. Pulses delivered beyond such upper bound can be auto-detected, though this can be adjusted by a TMS operator or another type of end-user. In the graphical interface 300, “Interval” indicates time between pulse trains. That time can be auto-detected or can be adjusted by a TMS operator or another type of end-user. The graphical interface 300 also can include a pane 304 that can include visual elements indicating if visible alerts are active (“ON”) or inactive (“OFF”), and a pane 308 that can include visual elements indicative if audible alerts are active (“ON”) or inactive (“OFF”). In some embodiments, a pane(s) similar to panes 304 and 308 can be included on a graphical interface presented at the monitoring device 110 (FIG. 1A).

FIG. 3B shows an example of a graphical interface 340 that one of the display devices 260(1) to 260(3) can display in connection with a TMS treatment. The graphical interface 340 presents the number of TMS pulses and trains that have already been delivered. The graphical interface 340 also includes a section 344 presenting a running timer that shows, for example, seconds remaining until the next pulse train. The graphical interface 340 also includes a progress bar 348 conveying a proportion 350 of a current treatment session that has been completed and another proportion 352 that remains to be completed.

FIG. 3C shows an example of a graphical interface 380 that one of the display devices 260(1) to 260(3) can display in connection with a TMS treatment. The graphical interface 380 presents a reading of a text file that can be contained on a memory card (such as a micro SD card) present in the treatment monitoring device 110. The memory card can store information about prior treatment sessions. In this example, the top record is clipped, but can be scrolled to show other the data. As is illustrated, the treatment record shows, in order: month, date, year, and day of week of the treatment, time the treatment started, total pulses administered, inter-train interval, and treatment duration in minutes and seconds. Additional information can be retained and displayed.

With further reference to FIG. 2B, the treatment monitoring device 110 can include buttons 262 to scroll through prior treatment recordings. The treatment monitoring device 110 also can include a color LED array 264 to convey, in some cases, visible alerts for the TMS operator. The treatment monitoring device 110 also can include actuation elements 266 (e.g., buttons and/or switches) to control the operation of the treatment monitoring device 110 and the treatment notification device 110.

The treatment monitoring device 110 also can include a rotary encoder 268. The rotary encoder 268 may be used to optionally adjust default settings for total pulses to be delivered, total trains, and inter-train intervals. The rotary encoder 268 may be programmable. The rotary encoder 268 may be programmed to that other parameters may be adjusted using the rotary encoder 268.

The treatment monitoring device 110 also defines an opening 270 to receive ambient sound 155 (FIG. 1) at an electret microphone or another type of audio input module. The treatment monitoring device 110, on sides 272 (e.g., lateral side and rear side), can further include a USB power port, programming port, and micro SD card slot.

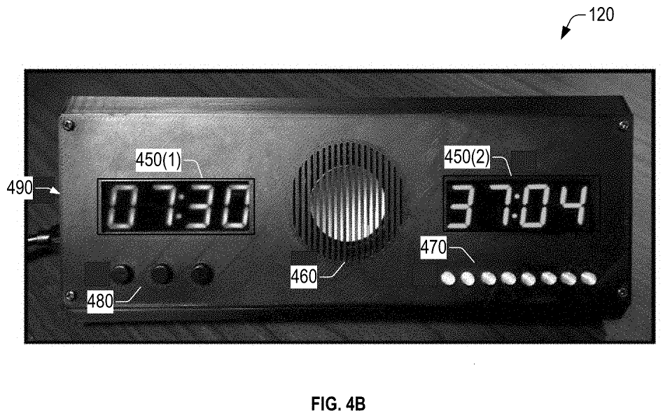

FIG. 4A shows an example of a treatment notification device 120, in accordance with one or more embodiments of this disclosure. The treatment notification device 120 includes a radio module 405 that can permit wireless communication between the notification device 120 and the treatment monitoring device 110. More specifically, the radio module 405 can receive signaling wirelessly from the treatment monitoring device 110 (not depicted in FIG. 4A).

The received data can identify various parameters of a TMS treatment session. Examples of those parameters include current number of TMS pulses administered as part of a TMS treatment session, time elapsed since commencement of the TMS treatment session, treatment duration, interval between sets of pulses, etc. The signaling can be embodied in, for example, various types of state messages that identify a current status of the TMS treatment session. In one example, a state message indicates that a next train of TMS pulses is about to be administered. In another example, a state message indicates that the TMS treatment session is about to end. In yet another example, a state message indicates that the TMS treatment session has ended. The notification device 120 can receive state messages asynchronously from the monitoring device 110. The signaling also can include, for example, configuration instructions, control instructions, and/or or other types of commands. To receive data and signaling wirelessly from the treatment monitoring device 110, the treatment notification device 120 includes a radio module 405. The radio module 405 can operate in a similar fashion as the radio module 240 integrated into the treatment monitoring device 110.

The treatment notification unit 120 also can include one or many processors 410. The processor(s) 410 can be arranged in numerous configurations depending at least on the specific complexity (functionality, operational capacity, etc.) of the notification device 120. Accordingly, the processor(s) 120 can be embodied in, or can constitute, a central processing unit (CPU); multiple CPUs; a graphics processing unit (GPU); multiple GPUs; a microprocessor or another type of digital signal processor; a programmable logic controller (PLC); an ASIC; a FPGA; other types of processing circuitry for executing program code or performing defined operations; a combination thereof; or similar.

The processor(s) 410 can control the operation of an audio output module 415 and at least one of multiple I/O interfaces 420. In some embodiments, the audio output module 415 can include a group of audio output devices (e.g., a headphone socket or piezoelectric speaker) and circuitry that permit generating and sending audio output signal (noise, tones, utterances, speech, music, and the like). Such circuitry can include, in some cases, digital-to-analog converters; volume control(s) and/or other audio controls.

In some embodiments, the I/O interface(s) 420 can include one or many display units 424 that can display various types of information pertaining to a TMS treatment session. The information can include, for example, a current time, time left in a treatment session, a visual element indicating the forthcoming application of a new pulse train, a visual element indicating termination of the treatment session, or a combination of the foregoing.

In some embodiments, at least one of the processor(s) 410 can be functionally coupled to a card reader/writer device 435 by means of a bus architecture 406. The card reader/writer device 435 can be functionally coupled to a card connector and interface 430. The card connector and interface 430 can constitute a card adapter (including an MMC slot, a SD slot, a SIM slot, or similar) to receive a memory card 425 of a defined form factor. The card adapter can permit functional connection to the bus architecture 406. A card slot defined by the card adapter is formed to receive the memory card 425. The memory card 425 can be connected to the notification device 120 by means of the card connector and interface 420. The card connector and interface 430 can be embodied in, for example, one of an MMC connector and interface, a SD connector and interface, and a SIM connector and interface. In other embodiments, at least one of the processor(s) 410 can be functionally coupled directly to the card connector and interface 430, without an intervening card reader/writer device 435. In those embodiments, one or several of the processor(s) 410 can read and write data to the memory card 425.

The card reader/writer device 435 can read data from the memory card 425. For example, the card reader/writer device 435 can read audio files retained in the memory card 425. The card reader/writer device 435 can retain a read audio file in one or more memory devices 445 (referred to as memory 445) included in the notification device 120. The audio files can be used for audible alerts. One or more of the audio files can be customized to a patient. The card reader/writer device 435 also can write other data to the memory card 425. The memory 445 can include one or more non-transitory storage media. In some embodiments, the memory 445 includes one or several solid-state memory devices.

At least one of the processor(s) 410 can execute an audio file to cause an audio output module 415 to emit audible sounds. In some configurations, the card reader/writer device 435 can read and one or several audio files from the memory card 425 in response to the memory card 425 being inserted into the notification device 120. After receiving a state message from the treatment monitoring device 110, at least one of the processor(s) 410 can respond by executing the audio file corresponding to the state message. As a result, the audio output module 415 can emit sound corresponding to the executed audio file. For example, the notification device 120 can receive a first state message indicating initiation of a next train of TMS pulses. In response, a processor of the processor(s) 410 can execute a first audio file, thus causing the audio output module 415 to emit sound corresponding to the executed first audio file. The notification device 120 also can receive a second state message indicating that the TMS session is about to end or has ended. In response, such a processor can execute a second sound file, thus causing the audio output module 415 to emit sound corresponding to the executed second audio file.

In addition, or in some configurations, the notification device 120 can response to a received state message by causing one or more of display unit(s) 424 included in I/O interfaces 420 to present a visible cue. More specifically, a display unit of the display unit(s) 424 can include one or several display devices oriented towards the chair 130 (FIG. 1), within LOS, in order to convey the information to a patient undergoing treatment. Examples of a display device include an LCD or LED display device, a seven-segment panel, or similar. Examples of the information conveyed to a patient can include current time, time left in the treatment session, and visual and/or audible alerts just prior to a train of pulses or at the conclusion of the session. The type and/or amount of information that is presented to the patient can be independently turned on or off per the patient's preference. Such a selection can be accomplished wirelessly, by means of signaling received from the treatment monitoring device 110, or by using switches located on the notification device 120.

In some embodiments, as is illustrated in FIG. 4B, the treatment notification unit 120 includes a first display unit 450(1) having multiple 7-segment LED display devices that can present a current time (optional). In those embodiments, the treatment notification unit 120 also includes a second display unit 450(2) also having multiple 7-segment LED display devices that can present time remaining in a treatment session, for example. Such a time can be presented in minutes and seconds (optional). The treatment notification device 120 also can include a speaker 460 that can emit an audible alert prior to a pulse train or at the end of a treatment session, or both. In addition, the treatment notification device 120 also can include a color LED array 470 that can present visible alerts prior to a pulse train or at the end of the treatment session, or both. Further, the treatment notification device 120 can include hardware switches and/or buttons to control the treatment notification device 120. These are not required for operation, as the treatment notification device 120 can be wirelessly controlled by means of the treatment monitoring device 110. The treatment notification device 120, on a side 490, can further include a USB power port, a programming port, and a microSD card slot (for sound files, for example).

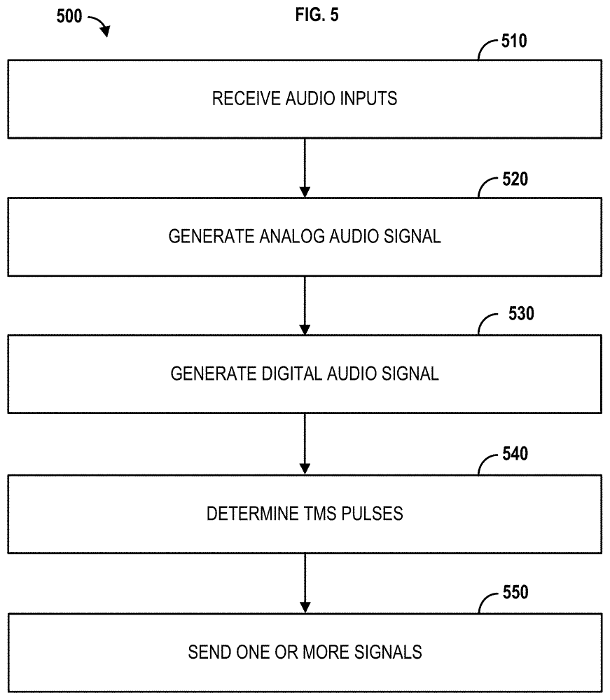

FIG. 5 shows an example method 500 executing on any of the devices described herein. At 510, one or more analog audio inputs associated with a TMS environment may be received. For example, the one or more analog audio inputs associated with the TMS environment may comprise TMS pulse sound (e.g., clicking sounds), ambient noise, or any other sounds. A pulse sound can measure up to 140 dB near the one or more coils 140 and may comprise one or more dominant frequency components in a range from approximately 2 kHz to about 5 kHz. For example, the treatment monitoring device may be configured to determine a characteristic sound profile of one or more TMS pulses of the plurality of TMS pulses. Based on the characteristic sound profile of the one or more TMS pulses, the treatment monitoring device may distinguish the sound of the TMS pulse from other sounds, such as ambient noise, speech, utterances, music, etc. Accordingly, the treatment monitoring device can detect the application of a TMS pulse by monitoring ambient sound within the treatment room. For example, the treatment monitoring device may be configured for essentially continuous audio processing of the ambient sound to determine if and when a TMS pulse has been emitted. By aurally detecting the one or more TMS pulses, the treatment monitoring device can determine and record several parameters of associated with individual TMS pulses and/or an entire treatment session comprising a series of TMS pulse trains.

At 520, at least one analog audio signal associated with the one or more analog audio inputs may be generated. The at least one analog audio signal may comprise for example, indications of amplitude, frequency, information associated with a time domain (e.g., intervals between one or more pulse trains, etc.).

At 530, at least one digital audio signal associated with the at least one analog audio inputs may be generated. Generating the at least one digital audio signal comprises performing an analog to digital conversion on the at least one analog audio signal associated with the one or more analog audio inputs. The at least one digital audio signal may comprise for example, indications of amplitude, frequency, information associated with a time domain (e.g., intervals between one or more pulse trains, etc.). For example, a microphone on a monitoring device is connected to microphone amplifier, then to a 10-bit analog-to-digital converter of a microcontroller. For example, sound samples of the environment may be continuously and/or periodically polled in various time increments (e.g., 3 millisecond epochs).

At 540, one or more TMS pulses may be determined. Determining the one or more TMS pulses may be based on the at least one digital audio signal, and by extension, based on the one or more analog audio inputs. The one or more TMS pulses may be determined by executing an audio processing algorithm. A maximum peak-to-peak soundwave intensity for each epoch may stored into a rolling array consisting of 3 epochs. To be considered a “possible TMS pulse”, the maximum sound from epoch 3 must be greater than the sum of those from epoch 1 and 2 by at least two-fold. The time for this occurrence may be recorded and/or stored. If a second and/or third such pulse is detected, a time interval between the pulses may be calculated. If the time interval between the pulses falls in a regular interval (within 10 milliseconds), the pulses may be considered to be from TMS. If the time interval between the pulses falls outside the regular interval (e.g., not within 10 millisecond), the pulses may be considered to not be from TMS. The pulses not considered to be from TMS may be considered as noise and/or otherwise be ignored. The time interval between pulses can be used to determine a pulse frequency. A total number of pulses in a “train” can be determined using the same method described above. If there is no additional TMS pulse sound after 2 seconds from the last pulse, the pulse train may be considered finished and recorded as a separate variable to keep track of pulse trains delivered. Similarly, a Fast Fourier Transform may be implemented to analyze the frequency of the sound as well as intensity. If a next set of TMS pulses (e.g., a next pulse train) is detected, a time interval may calculated and an intertrain interval can be determined. Once the intertrain interval is known, the time left in treatment can be simply calculated and displayed (assuming a default of 3000 pulses/session, but can be adjusted by the user). If the operator pauses the treatment and a pulse is not detected after the expected intertrain interval, the monitor will assume the treatment has been paused and stop the countdown timer appropriately. If there is no pulse after a prolonged period (˜5 minutes), the treatment will be considered terminated and the treatment parameters will be appended into a text file on the microSD card.

At 550, one or more signals may be sent. For example, the one or more signals may be sent from the treatment monitoring device to a display device. For example, the one or more signals may be indicative of an arrival of a subset of TMS pulses of the one more TMS pulses. For example, the one or more signals may comprise the TMS data and other TMS information and may be output audibly and/or visually for observation by a patient or TMS operator. For example, a wall-mounted unit may be configured to receive the one or more signals and output visual and/or audible alerts to the patient regarding the status of the TMS treatment, such as time left in treatment, or notification of an upcoming set of TMS pulses to reduce startling the patient For example, for subsequent pulse trains, the monitoring device can display a countdown to the next pulse train (as shown on FIG. 3b) and also send a radio signal to the patient notification device such that an audible or visible alert can be directed at the patient a couple seconds before the next pulse train is delivered (optionally). For example, the treatment monitoring technologies can determine date/time of treatment, total number of pulses administered, treatment duration, interval between sets of pulses, etc. The monitoring device may be configured to facilitate manual control for starting, pausing, and stopping a treatment cycle. The monitoring device may be configured to facilitate changing a default total number of pulses and an intertrain interval (e.g., a time between pulses and/or trains of pulses).

The method may comprise determining one or more TMS pulse parameters associated with the one or more TMS pulses wherein the one or more TMS pulse parameters comprise one or more of: a treatment date, a treatment start time, a number of pulses delivered, an inter-train interval, a duration of treatment, a time until a next pulse train, a time left in treatment, a treatment end time, one or more combinations thereof, and the like. The method may comprise determining, based on the one or more TMS pulses, a quantity of TMS pulses up to a current time. The method may comprise determining a time interval elapsed since a first TIMS pulse of the one or more TMS pulses. The method may comprise sending, to a display device, a signal indicative of the quantity of TMS pulses and the time interval, wherein the signal indicative of the quantity of TMS pulses and the time interval is configured to cause the display device to output the a value associated with the quantity of TMS pulses and a value associated with the time interval.

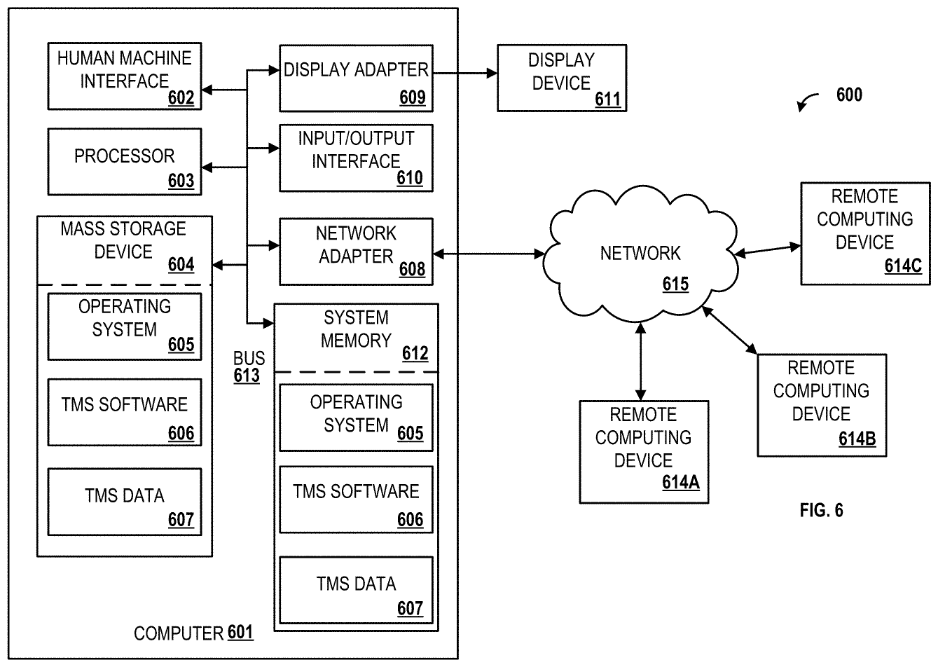

FIG. 6 shows an example computing environment 600. The above described disclosure may be implemented on a computer 601 as illustrated in FIG. 6 and described below. FIG. 6 is a block diagram illustrating an example operating environment for performing the disclosed methods. This example operating environment is only an example of an operating environment and is not intended to suggest any limitation as to the scope of use or functionality of operating environment architecture. Neither should the operating environment be interpreted as having any dependency or requirement relating to any one or combination of components illustrated in the example operating environment.

The present disclosure can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Examples comprise set top boxes, programmable consumer electronics, network PCs, minicomputers, mainframe computers, distributed computing environments that comprise any of the above systems or devices, and the like.

The processing of the disclosed can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 601. The components of the computer 601 can comprise, but are not limited to, one or more processors 603, a system memory 612, and a system bus 613 that couples various system components including the one or more processors 603 to the system memory 612. The system can utilize parallel computing.

The system bus 613 represents one or more of several possible types of bus structures, including a memory bus or memory controller, a peripheral bus, an accelerated graphics port, or local bus using any of a variety of bus architectures. By way of example, such architectures can comprise a Peripheral Component Interconnects (PCI), a PCI-Express bus, Universal Serial Bus (USB), hypertransport and other current high speed motherboard buses, and the like. The bus 613, and all buses specified in this description can also be implemented over a wired or wireless network connection and each of the subsystems, including the one or more processors 603, a mass storage device 604, an operating system 605, tagging software 606, tagging data 607, a network adapter 608, the system memory 612, an Input/Output Interface 610, a display adapter 609, a display device 611, and a human machine interface 602, can be contained within one or more remote computing devices 614A, 614B, 614C at physically separate locations, connected through buses of this form, in effect implementing a fully distributed system.

The computer 601 typically comprises a variety of computer readable media. Example readable media can be any available media that is accessible by the computer 601 and comprises, for example and not meant to be limiting, both volatile and non-volatile media, removable and non-removable media. The system memory 612 comprises computer readable media in the form of volatile memory, such as random access memory (RAM), and/or non-volatile memory, such as read only memory (ROM). The system memory 612 typically contains data such as the tagging data 607 and/or program modules such as the operating system 605 and the tagging software 606 that are immediately accessible to and/or are presently operated on by the one or more processors 603.

The computer 601 can also comprise other removable/non-removable, volatile/non-volatile computer storage media. By way of example, FIG. 6 illustrates the mass storage device 604 which can facilitate non-volatile storage of computer code, computer readable instructions, data structures, program modules, and other data for the computer 601. For example and not meant to be limiting, the mass storage device 604 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 604, including by way of example, the operating system 605 and the tagging software 606. Each of the operating system 605 and the tagging software 606 (or some combination thereof) can comprise elements of the programming and the tagging software 606. The tagging data 607 can also be stored on the mass storage device 604. The tagging data 607 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

The user or device can enter commands and information into the computer 601 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the one or more processors 603 via the human machine interface 602 that is coupled to the system bus 613, but can be connected by other interface and bus structures, such as a parallel port, game port, an IEEE 1394 Port (also known as a Firewire port), a serial port, or a universal serial bus (USB), a wireless peripheral connection such as, for example, Bluetooth, WiFi, and/or Ultra-wideband (UWB).

The display device 611 can also be connected to the system bus 613 via an interface, such as the display adapter 609. It is contemplated that the computer 601 can have more than one display adapter 609 and the computer 901 can have more than one display device 911. For example, the display device 611 can be a monitor, an LCD (Liquid Crystal Display), or a projector. In addition to the display device 611, other output peripheral devices can comprise components such as speakers (not shown) and a printer (not shown) which can be connected to the computer 601 via the Input/Output Interface 610. Any step and/or result of the methods can be output in any form to an output device. Such output can be any form of visual representation, including, but not limited to, textual, graphical, animation, audio, tactile, and the like. The display device 611 and computer 601 can be part of one device, or separate devices.

The computer 601 can operate in a networked environment using logical connections to one or more remote computing devices 614A, 614B, 614C. By way of example, a remote computing device can be a personal computer, portable computer, smartphone, a server, a router, a network computer, a peer device or other common network node, and so on. Logical connections between the computer 601 and a remote computing device 614A, 614B, 614C can be made via a network 615, such as a local area network (LAN) and/or a general wide area network (WAN). Such network connections can be through the network adapter 608. The network adapter 608 can be implemented in both wired and wireless environments. Such networking environments are conventional and commonplace in dwellings, offices, enterprise-wide computer networks, intranets, and the Internet.

For purposes of illustration, application programs and other executable program components such as the operating system 605 are illustrated herein as discrete blocks, although it is recognized that such programs and components reside at various times in different storage components of the computing device 601, and are executed by the one or more processors 603 of the computer. An implementation of the selective tagging software 606 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Example computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

While the technologies (e.g., techniques, computer program products, devices, and systems) of this disclosure have been described in connection with various embodiments and specific examples, it is not intended that the scope be limited to the particular embodiments put forth, as the embodiments herein are intended in all respects to be illustrative rather than restrictive

As used in this application, the terms “environment,” “system,” “module,” “component,” “architecture,” “interface,” “unit,” and the like are intended to encompass an entity that includes either hardware, software, or a combination of hardware and software. Such an entity can be embodied in, or can include, for example, a signal processing device. In another example, the entity can be embodied in, or can include, an apparatus with a defined functionality provided by optical parts, mechanical parts, and/or electronic circuitry. The terms “environment,” “system,” “engine,” “module,” “component,” “architecture,” “interface,” and “unit” can be utilized interchangeably and can be generically referred to functional elements.

A component can be localized on one processing device or distributed between two or more processing devices. Components can communicate via local and/or remote architectures in accordance, for example, with a signal (either analogic or digital) having one or more data packets (e.g., data from one component interacting with another component in a local processing device, distributed processing devices, and/or across a network with other systems via the signal).

As yet another example, a component can be embodied in or can include an apparatus with a defined functionality provided by mechanical parts operated by electric or electronic circuitry that is controlled by a software application or firmware application executed by a processing device. Such a processing device can be internal or external to the apparatus and can execute at least part of the software or firmware application. Still in another example, a component can be embodied in or can include an apparatus that provides defined functionality through electronic components without mechanical parts. The electronic components can include signal processing devices to execute software or firmware that permits or otherwise facilitates, at least in part, the functionality of the electronic components. For the sake of illustration, an example of such processing device(s) includes an integrated circuit (IC), an application-specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed or otherwise configured (e.g., manufactured) to perform the functions described herein.

In some embodiments, components can communicate via local and/or remote processes in accordance, for example, with a signal (either analog or digital) having one or more data packets (e.g., data from one component interacting with another component in a local system, distributed system, and/or across a network such as a wide area network with other systems via the signal). In addition, or in other embodiments, components can communicate or otherwise be coupled via thermal, mechanical, electrical, and/or electromechanical coupling mechanisms (such as conduits, connectors, combinations thereof, or the like). An interface can include input/output (I/O) components as well as associated processors, applications, and/or other programming components.

Conditional language, such as, among others, “can,” “could,” “might,” or “may,” unless specifically stated otherwise, or otherwise understood within the context as used, is generally intended to convey that certain implementations could include, while other implementations do not include, certain features, elements, and/or operations. Thus, such conditional language generally is not intended to imply that features, elements, and/or operations are in any way required for one or more implementations or that one or more implementations necessarily include logic for deciding, with or without user input or prompting, whether these features, elements, and/or operations are included or are to be performed in any particular implementation.

The flowchart and block diagrams in the Figures illustrate the architecture, functionality, and operation of possible implementations of examples of systems, methods, and computer program products according to various embodiments of the present disclosure. In this regard, each block in the flowchart or block diagrams may represent a module, segment, or portion of instructions, which includes one or more machine-executable or computer-executable instructions for implementing the specified operations. It is noted that each block of the block diagrams and/or flowchart illustration, and combinations of blocks in the block diagrams and/or flowchart illustration, can be implemented by special purpose hardware-based devices that perform the specified functions or operations or carry out combinations of special purpose hardware and computer instructions.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

What has been described herein in the present specification and annexed drawings includes examples of systems, apparatuses, devices, and techniques for monitoring of a TMS treatment session and notification of parameters pertinent to the treatment session. It is, of course, not possible to describe every conceivable combination of components and/or methods for purposes of describing the various elements of the disclosure, but it can be recognized that many further combinations and permutations of the disclosed elements are possible. Accordingly, it may be apparent that various modifications can be made to the disclosure without departing from the scope or spirit thereof. In addition, or as an alternative, other embodiments of the disclosure may be apparent from consideration of the specification and annexed drawings, and practice of the disclosure as presented herein. It is intended that the examples put forth in the specification and annexed drawings be considered, in all respects, as illustrative and not limiting. Although specific terms are employed herein, they are used in a generic and descriptive sense only and not for purposes of limitation.

The disclosure can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

While the disclosure has been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as an example only, with a true scope and spirit being indicated by the following claims.

What is claimed is:

1. A method comprising:
receiving one or more analog audio inputs associated with a transcranial magnetic stimulation (TMS) environment;
generating, based on the one or more analog audio inputs, at least one analog audio signal associated with the one or more analog audio inputs;
generating, based on the at least one analog audio signal associated with the one or more analog audio inputs, at least one digital audio signal;
determining, based on the at least one digital audio signal, one or more TMS pulses; and
sending, based on the one or more TMS pulses, to a second computing device, one or more signals.

2. The method of claim 1, wherein the one or more signals are indicative of an arrival of a subset of TMS pulses of the one more TMS pulses.

3. The method of claim 1, wherein the one or more analog audio inputs comprise TMS pulse sounds originating from one or more TMS coils.

4. The method of claim 1, wherein generating the at least one digital audio signal comprises performing an analog to digital conversion on the at least one analog audio signal associated with the one or more analog audio inputs.

5. The method of claim 1, further comprising determining one or more TMS pulse parameters associated with the one or more TMS pulses wherein the one or more TMS pulse parameters comprise one or more of: a treatment date, a treatment start time, a number of pulses delivered, an inter-train interval, a duration of treatment, a time until a next pulse train, a time left in treatment, a treatment end time, one or more combinations thereof, and the like.

6. The method of claim 1, further comprising:
determining, based on the one or more TMS pulses, a quantity of TMS pulses up to a current time;
determining, a time interval elapsed since a first TIMS pulse of the one or more TMS pulses; and
sending, to a display device, a signal indicative of the quantity of TMS pulses and the time interval, wherein the signal indicative of the quantity of TMS pulses and the time interval is configured to cause the display device to output the a value associated with the quantity of TMS pulses and a value associated with the time interval.

7. The method of claim 6, wherein the display device is configured to receive the signal indicative of the quantity of TMS pulses and the time interval and is further configured to output one or more visible cues indicative of the quantity of TMS pulses and one or more visible cues indicative of the time interval.

8. An apparatus comprising:
one or more processors; and
memory storing processor executable instructions that, when executed by the one or more processors, cause the apparatus to:
receive one or more analog audio inputs associated with a transcranial magnetic stimulation (TMS) environment;
generate, based on the one or more analog audio inputs, at least one analog audio signal associated with the one or more analog audio inputs;
generate, based on the at least one analog audio signal associated with the one or more analog audio inputs, at least one digital audio signal;
determine, based on the at least one digital audio signal, one or more TMS pulses; and
send, based on the one or more TMS pulses, to a second computing device, one or more signals.

9. The apparatus of claim 8, wherein the one or more signals are indicative of an arrival of a subset of TMS pulses of the one more TMS pulses.

10. The apparatus of claim 8, wherein the one or more analog audio inputs comprise TMS pulse sounds originating from one or more TMS coils.

11. The apparatus of claim 8, wherein the processor executable instructions that, when executed by the one or more processors, cause the apparatus to generate the at least one digital audio signal, further cause the apparatus to perform an analog to digital conversion on the at least one analog audio signal associated with the one or more analog audio inputs.

12. The apparatus of claim 8, wherein the processor executable instructions, when executed by the one or more processors, further cause the apparatus to determining one or more TMS pulse parameters associated with the one or more TMS pulses wherein the one or more TMS pulse parameters comprise one or more of: a treatment date, a treatment start time, a number of pulses delivered, an inter-train interval, a duration of treatment, a time until a next pulse train, a time left in treatment, a treatment end time, one or more combinations thereof, and the like.

13. The apparatus of claim 8, wherein the processor executable instructions, when executed by the one or more processors, further cause the apparatus to:
determine, based on the one or more TMS pulses, a quantity of TMS pulses up to a current time;
determine, a time interval elapsed since a first TIMS pulse of the one or more TMS pulses; and
send, to a display device, a signal indicative of the quantity of TMS pulses and the time interval, wherein the signal indicative of the quantity of TMS pulses and the time interval is configured to cause the display device to output the a value associated with the quantity of TMS pulses and a value associated with the time interval.

14. The apparatus of claim 13, wherein the display device is configured to receive the signal indicative of the quantity of TMS pulses and the time interval and is further configured to output one or more visible cues indicative of the quantity of TMS pulses and one or more visible cues indicative of the time interval.

15. A system, comprising:
a first computing device configured to:
receive one or more analog audio inputs associated with a transcranial magnetic stimulation (TMS) environment;
generate, based on the one or more analog audio inputs, at least one analog audio signal associated with the one or more analog audio inputs;
an audio processing device configured to:
receive the at least one analog audio signal associated with the one or more analog audio inputs;
generate, based on the at least one analog audio signal associated with the one or more analog audio inputs, at least one digital audio signal;
determine, based on the at least one digital audio signal, one or more TMS pulses; and
a radio module configured to send, based on the one or more TMS pulses, to a second computing device, one or more signals.

16. The system of claim 15, wherein the one or more signals are indicative of an arrival of a subset of TMS pulses of the one more TMS pulses.

17. The system of claim 15, wherein the one or more analog audio inputs comprise TMS pulse sounds originating from one or more TMS coils.

18. The system of claim 15, wherein the first computing device is configured to generate the at least one digital audio signal by performing an analog to digital conversion on the at least one analog audio signal associated with the one or more analog audio inputs.

19. The system of claim 15, wherein the first computing device is further configured to determine one or more TMS pulse parameters associated with the one or more TMS pulses wherein the one or more TMS pulse parameters comprise one or more of: a treatment date, a treatment start time, a number of pulses delivered, an inter-train interval, a duration of treatment, a time until a next pulse train, a time left in treatment, a treatment end time, one or more combinations thereof, and the like.

20. The system of claim 15, wherein the first computing device is further configured to:
determine, based on the one or more TMS pulses, a quantity of TMS pulses up to a current time;
determine, a time interval elapsed since a first TIMS pulse of the one or more TMS pulses; and
send, to a display device, a signal indicative of the quantity of TMS pulses and the time interval, wherein the signal indicative of the quantity of TMS pulses and the time interval is configured to cause the display device to output the a value associated with the quantity of TMS pulses and a value associated with the time interval.
